(19) United States
(12) Patent Application Publication
(10) Pub. No.: US 2014/0011980 A1
(43) Pub. Date: Jan. 9, 2014

(54) METHODS FOR STERILIZING COMPOSITIONS AND RESULTING COMPOSITIONS

(71) Applicant: Allergan, Inc., Irvine, CA (US)

(72) Inventors: Kaustubh S. Chitre, Goleta, CA (US); Xiaojie Yu, Irvine, CA (US); Nicholas J. Manesis, Escondido, CA (US)

(21) Appl. No.: 13/934,976

(22) Filed: Jul. 3, 2013

Related U.S. Application Data
(60) Provisional application No. 61/667,701, filed on Jul. 3, 2012.
(60) Provisional application No. 61/692,609, filed on Aug. 23, 2012.
(60) Provisional application No. 61/704,990, filed on Sep. 24, 2012.

(51) Int. Cl. A61L 2/08

(52) U.S. Cl. CPC A61L 2/08
USPC 530/356; 422/24

(57) ABSTRACT

Method for sterilizing a hydrogel composition include subjecting the composition to pulsed light comprising broadband spectrum radiation, the pulsed light being at a dose effective to sterilize the composition without causing significant change in rheology of the composition.

This application claims priority to U.S. Provisional Patent Application Nos. 61/667,701, filed Jul. 3, 2013; 61/692,609, filed Aug. 23, 2012; and 61/704,990, filed Sep. 24, 2012, the entire contents of which are hereby incorporated by reference.

### BACKGROUND

This invention generally relates to methods for sterilizing hydrogel compositions, and more specifically relates to methods for sterilizing polymer and protein based compositions, for example, but not limited to, biomaterials useful for augmenting or reconstructing human soft tissue, for example, dermal fillers and other soft tissue fillers.

Many biomaterial compositions are being developed and are in commercial use which requires sterilization, that is, destruction of attenuation to a harmless nature of unwanted biologic material such as pathogens, microbes of bacteria, and prior to the administration of the composition by injection or implantation into a human patient. Such compositions, for example, include those useful as implantable materials for bulking or contouring tissue in cosmetic and reconstructive procedures, or as implantable vehicles for delivering active pharmaceuticals or drugs into a patient. Many such compositions are polymer based. These compositions include materials such as hyaluronic acid (HA), alginic acid, cellulose, collagen, elastin, and gelatin. Proteins, polysaccharides and carbohydrates in these materials are susceptible to molecular breakdown when exposed to conventional heat temperature sterilization procedures, such as autoclave, or when subjected to ionizing radiation such as gamma radiation. Conventionally, many of these energy-sensitive biomaterials are sterilized in bulk by microfiltration processes which are intended to physically remove microbes from the compositions. The filtered compositions must then be packaged in syringes and/or vials for use by physicians. These conventional microfiltration processes are expensive and time consuming.

Hence, there remains a need for improved sterilization methods for biomaterials intended for administration to a human being.

### SUMMARY

The present invention meets this and other needs by providing methods for sterilizing compositions, for example, hydrogel compositions, for example, injectable hydrogel compositions, for example, injectable hydrogels comprising crosslinked biopolymers. The method generally comprises the step of subjecting the composition to a dose of broadband spectrum radiation effective to inactivate pathogen, microbes and other microorganisms. More particularly, the method comprises subjecting the composition to pulsed radiation, hereinafter sometimes pulsed light, comprising broadband spectrum radiation. The broadband spectrum radiation may have a band range from about 100 nm to about 1100 nm wavelength. The broadband spectrum radiation includes wavelengths in the ultraviolet range, the visible light range and the infrared range. In some embodiments, has a wavelength distribution of about 54% UV wavelengths, 26% visible wavelengths and about 20% infrared wavelengths. This form of radiation may be provided by a Xenon lamp.

The pulsed light is effective to sterilize the composition, that is, inactivate microorganisms and microbes in the composition, for example, throughout the composition, without causing significant deterioration of the composition, for example, without causing significant change in rheology of the composition.

In one embodiment, the pulsed light has an energy defined by a UV fluence at 254 nm of between about 100 mJ/sqcm to about 2000 mJ/sqcm, for example, between about 300 mJ/sqcm to about 1800 mJ/sqcm.

In a specific embodiment, the pulsed light has an energy defined by a UV fluence at 254 nm of between about 700 mJ/sqcm to about 800 mJ/sqcm. In another embodiment, the pulsed light has an energy defined by a UV fluence at 254 nm of between about 1400 mJ/sqcm to about 1600 mJ/sqcm.

In some embodiment, the pulsed light has a pulse frequency of between about 1 pulse per second to about 10 pulses per second, for example, about 3 pulses per second.

In yet another aspect of the invention, the composition is subjected to the pulsed light for a time period of no greater than 240 seconds. In one embodiment, the composition is subjected to the pulsed light for a time period of no greater than 120 seconds. In one embodiment, the composition is subjected to the pulsed light for a time period of no greater than 40 seconds. In one embodiment, the composition is subjected to the pulsed light for a time period of no greater than 30 seconds. In one embodiment, the composition is subjected to the pulsed light for a time period of no greater than 20 seconds. In one embodiment, the composition is subjected to the pulsed light for a time period of 10 seconds. In one embodiment, the composition is subjected to the pulsed light for a time period of 5 seconds. In yet another embodiment, the composition is subjected to the pulsed light for a time period of no greater than one second.

In still a further aspect of the invention, the composition comprises collagen. In another aspect, the composition comprises hyaluronic acid (HA). In a specific embodiment, the composition comprises hyaluronic acid and collagen, for example, crosslinked hyaluronic acid and collagen. In another specific embodiment, the composition may be in a form of a hydrogel product comprising hyaluronic acid crosslinked to collagen, the product being suitable for combining with extracted adipose tissue, the combination being useful in augmenting or reconstructing human soft tissue, for example, in fat grafting procedures.

In another aspect of the invention, the pulsed light is effective to sterilize the composition without raising the temperature of the composition more than 90 degrees C. In some embodiments, the pulsed light is effective to sterilize the composition without raising the temperature of the composition more than 20 degrees C. In other embodiments, the dose is effective to sterilize the composition without raising the temperature of the composition more than 15 degrees C., for example, more than 10 degrees C., for example, more than 5 degrees C.

In yet another aspect, the pulsed light is effective to sterilize the composition with a loss in rheology (G′/G″) of less than about 10%, or less than about 8%, or less than about 5%.

Further provided is a product comprising crosslinked hyaluronic acid and collagen, sterilized by the methods described herein. The product may be useful for combining with adipose tissue for use in fat grafting procedures. The product may comprise a composition comprising crosslinked hyaluronic acid and collagen, the composition having been sterilized by subjecting the composition to a dose of broadband spectrum radiation, for example, pulsed light comprising broadband spectrum radiation having a band range from about 100 nm to about 1100 nm wavelength, wherein the pulsed light is effective to sterilize the composition without causing significant deterioration of the composition, for example, without causing any significant change or deterioration in rheology of the composition.

The product may further comprise a vial or syringe containing the composition. In some embodiments, the composition has been so subjected to the pulsed light while the composition was contained in the vial or the syringe.

### BRIEF DESCRIPTION OF THE DRAWINGS

Further aspects and/or advantages of the present invention may be more thoroughly understood and/or appreciated with reference to the following Detailed Description and accompanying Drawings of which:

FIG. 1 is a line graph showing shows a wavelength spectrum of broadband radiation from a pulsed Xenon lamp, showing, wavelength in nanometers (nm) along the x-axis, in terms of absolute irradiance, along the y-axis, this broadband radiation being useful in certain aspects of the invention;

FIG. 2 is a response spectrum for the detector used for measuring lamp output, showing peak wavelength of 254 nm;

FIG. 3 is a photograph of test Soy-Agar plates used in an experiment testing the effectiveness of one of the presently described embodiments of the invention;

FIG. 4 is a graph comparing an untreated hydrogel (hyaluronic acid and collagen-based) and a pulsed light-treated hydrogel in terms of changes in rheology;

FIG. 5 is a plot showing E Coli inactivation (viable count) along the y-axis, versus treatment time, along the x-axis, in an a hydrogel treated with an embodiment of the invention;

FIG. 6 is a plot showing Geobacilus stearothermophilus spore inactivation (viable count) along the y-axis, versus treatment time, along the x-axis, in an a hydrogel treated with an embodiment of the invention;

FIG. 7 is a line graph showing temperature over time of a hydrogel during treatment with an embodiment of the invention, as well as temperature over time of the ambient air, the packaging of the hydrogel, and the shelf on which the packaging was placed during the treatment;

FIG. 8 shows a linear plot of temperature increase over time for multiple hydrogels during pulsed light treatment in accordance with embodiments of the invention; and

FIG. 9 is a graph comparing, in terms of changes in rheology (G′ and G″), a hydrogel treated with pulsed light treatment of the invention, an untreated hydrogel, and a hydrogel treated with conventional heat sterilization (e.g. autoclave).

### DETAILED DESCRIPTION

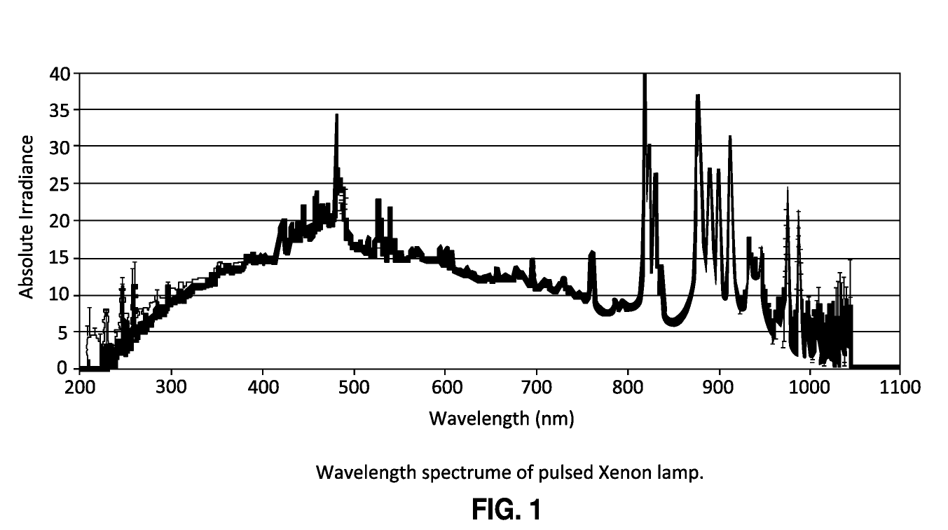

Methods for sterilizing compositions are provided which generally comprise the step of subjecting the composition to a dose of broadband spectrum radiation. More particularly, the methods comprise subjecting the composition to pulsed radiation, or pulsed light comprising broadband spectrum radiation. A wavelength spectrum of broadband spectrum radiation suitable for the present methods is shown in FIG. 1. The broadband spectrum radiation includes wavelengths in the ultraviolet range, the visible light range and the infrared range. In a specific embodiment, the radiation is provided by a Xenon lamp and may have a band range from about 100 nm to about 1100 nm.

In accordance with the invention, the pulsed light is effective to sterilize the composition, that is, inactivate pathogens, microbes and other microorganisms in the composition, without causing significant deterioration, for example, without causing significant changes in rheological properties of the composition. When exposed to the pulsed light as will be described in greater detail herein, it is believed that DNA of microorganisms in the composition undergoes rearrangement. The radiation passes through bacterial cells and destroys cell walls, making microorganisms ineffective to reproduce.

The composition sterilized by the present methods may comprise various biopolymers. In one embodiment, the composition comprises collagen. In another aspect, the composition comprises hyaluronic acid. In a specific embodiment, the composition comprises hyaluronic acid and collagen, for example, crosslinked hyaluronic acid and collagen.

Hyaluronic acid is a non-sulfated glycosaminoglycan that enhances water retention and resists hydrostatic stresses. Hyaluronic acid herein may include its fully protonated, or nonionic form, as well as any anionic forms and salts of hyaluronic acid, such as sodium salts, potassium salts, lithium salts, magnesium salts, calcium salts, etc.

Collagen is a protein that forms fibrils and sheets that bear tensile loads. Collagen also has specific integrin-binding sites for cell adhesion and is known to promote cell attachment, migration, and proliferation. Collagen may be positively charged because of its high content of basic amino acid residues such as arginine, lysine, and hydroxylysine. Reference to collagen herein may include uncharged collagen, as well as any cationic forms, anionic forms, or salts of collagen.

The compositions can include, alternatively or additionally, other biopolymers, for example, cellulose, chitosan, and chondroitin.

In a more specific aspect of some embodiments of the invention, the composition may be in a form of a hydrogel product comprising hyaluronic acid crosslinked to collagen, the product being suitable for combining with extracted adipose tissue, the combination being useful in augmenting or reconstructing human soft tissue, for example, in fat grafting procedures. The composition may be a hydrogel in the form of a crosslinked macromolecular matrix synthesized by coupling a hyaluronic acid with a collagen using a coupling agent, such as a carbodiimide. Hyaluronic acid may serve as a biocompatible water-binding component, providing bulk and isovolumetric degradation. Additionally, collagen may impart cell adhesion and signaling domains to promote cell attachment, migration, and other cell functions such as extra-cellular matrix deposition. These compositions can be made to be injectable for minimally invasive implantation through syringe and needle.

Advantageously, the methods of the present invention are useful for sterilizing these compositions while they are contained in a vial, syringe, or other end-user container, wherein the end-user in this case being a physician, doctor or technician who will be treating a patient with the product. This eliminates the complications associated with sterilizing these materials in bulk and then transferring the materials to individual syringes or vials in their sterile form.

In one embodiment, the composition is one or more of the hydrogel compositions described in commonly owned U.S. patent application Ser. No. 61/586,589, filed on Jan. 13, 2012, the entire disclosure of which is incorporated herein by this specific reference.

In accordance with one aspect of the invention, the step of subjecting the composition comprises subjecting the composition to pulsed light comprising broadband spectrum radiation such as characterized in FIG. 1, wherein the pulsed light has an energy between about 100 mJ/sqcm to about 2000 mJ/sqcm, for example, the pulsed light may have an energy between about 300 mJ/sqcm to about 1800 mJ/sqcm, when measured at a UV fluence of 254 nm.

In a specific embodiment, the pulsed light is provided, at UV fluence of 254 nm, at between about 700 mJ/sqcm to about 800 mJ/sqcm. In another embodiment, the pulsed light is provided, at UV fluence of 254 nm, between about 1400 mJ/sqcm to about 1600 mJ/sqcm.

The pulsed light may have a suitable pulse frequency for providing the desired energy level to the composition, for example, the pulsed light may have a pulse frequency of between one pulse per second to about 10 pulses per second or more. In one embodiment, the pulse frequency is about 3 pulses per second.

The composition is subjected to the pulsed light for a time period of no greater than 240 seconds, no greater than 120 seconds, no greater than 40 seconds, or no greater than 30 seconds. In some embodiments, the composition is subjected to the pulsed light for a time period of no greater than 10 seconds, no greater than 5 seconds, or no greater than one second.

In another aspect of the invention, the pulsed light is effective to sterilize the composition without raising the temperature of the composition to a level which may cause degradation or other undesirable change in the composition. Depending upon the specific composition being sterilized, in some embodiments, the methods are effective to sterilize the composition without changing the temperature by more than about 90 degrees C. In other embodiments in which the composition is relatively more temperature sensitive, the pulsed light is effective to sterilize the composition without raising the temperature of the composition more than about 20 degrees C. In yet other embodiments, the dose is effective to sterilize the composition without raising the temperature of the composition more than about 15 degrees C., for example, more than about 10 degrees C., for example, or more than about 5 degrees C.

Further provided are methods of sterilizing injectable, or implantable compositions, such as HA based or HA/Collagen based hydrogels, using pulsed broadband spectrum radiation, wherein the effective sterilizing dose of the radiation retains the rheology of the hydrogel. In some embodiments, the methods are effective to sterilize the hydrogel with a loss in rheology (G′/G″) of less than about 10%, or less than about 8%, or less than about 5%.

Further provided is a product that includes a composition which has been sterilized by the presently described methods. In a specific embodiment, the product comprises a hyaluronic acid collagen hydrogel useful for combining with adipose tissue for use in fat grafting procedures. The product may comprise a composition comprising crosslinked hyaluronic acid and collagen, the composition having been sterilized by subjecting the composition to pulsed light comprising broadband spectrum radiation, for example, radiation having a band range from about 100 nm to about 1100 nm, wherein the dose is effective to sterilize the composition without causing significant deterioration of the composition.

The product may further comprise a vial or syringe containing the composition. In some embodiments, the composition has been so subjected to the sterilizing dose of broadband spectrum radiation while the composition was contained in the vial or the syringe.

### EXAMPLE 1

### Hyaluronic Acid and Collagen-based Hydrogel Material

The hydrogel material tested in this experiment was an experimental hydrogel comprising HA and collagen, specifically, HA and collagen chemically crosslinked to form a hydrogel (hereinafter sometimes “HA-Coll gel”). The hydrogel had a concentration of about 12 mg/ml of HA and about 6 mg/ml Collagen.

Clear transparent high density polyethylene (HDPE) bags were selected for packaging the hydrogels during the sterilization process.

Geobacilus stearothermophilus spores and EColi O157:H7 vegetative cells were selected as microorganisms to study for effectiveness of the present methods.

The experimental design consisted of following conditions:

Microorganisms (E. coli 0157:H7 cells and G. sterarothermophilus spores)×2 treatment times (20sec and 40 sec)p×1 distance (3.26″ from the quartz window)×1 hydrogel formulation×3 replications=12 treatments+6 controls=18 samples.

These samples were subjected to pulsed light having broadband spectrum radiation between 100 nm and 1100 nm wavelength with approximate UV-54%, visible-26% and IR-20% distribution.

Equipment used was a SteriPulse-XL 3000 bench-top sterilization equipment available from Xenon Corporation, (Boston, Mass.).

The equipment includes a central processing unit (CPU) and a sterilization chamber. The CPU is configured to control the power, pulse time and sterilization parameters. The chamber includes a lamp housing and loading tray for samples. The lamp is Xenon UV source with polychromatic output 100 nm and 1100 nm wavelength. The lamp generated 360 microsecond pulses. The lamp was pulsed at 3 pulses per second.

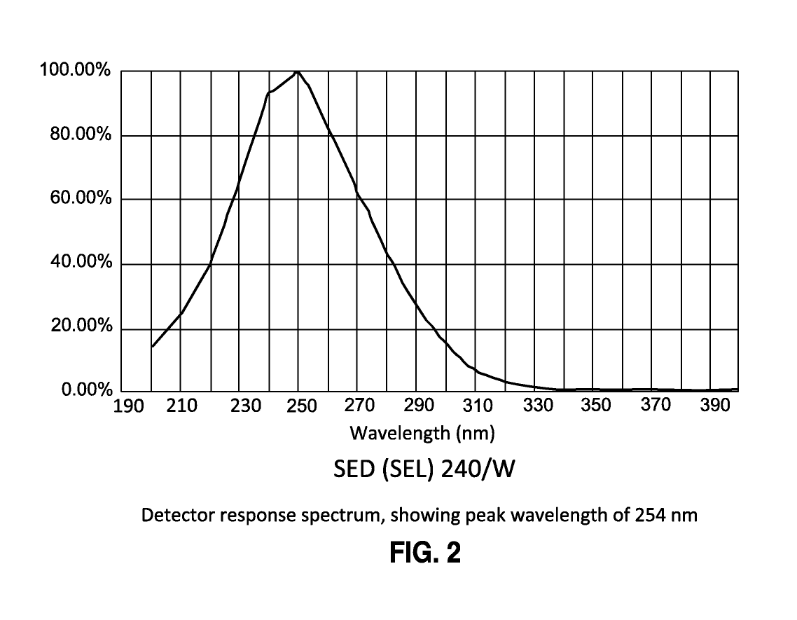

FIG. 2 shows a detector response spectrum having a peak wavelength of 254 nm. Lamp output was measured with UV-photodiode sensor, for example, a SED240 UV sensor and an ILT radiation meter available from International Lights Inc. (Peabody, Mass.). The ILT radiation meter gives UV fluence readings in mJ/sq.cm.

The pulsed light proved to be effective against Escherichia coli 0157:H7. A 20-sec treatment with pulsed light resulted in 6.98±0.00 log₁₀ CFU/g reduction. No survival was observed at all the tested treatment conditions (20 and 40 sec).

TABLE 1
Survival of microorganisms in HA-Coll gel
after pulsed light method of the invention

| | Escherichia coli | Geobacillus |
|---|---|---|
| Treatment | O157:H7 cells | stearothermophillus |
| Time Sec | (log₁₀ CFU/g) | spores (log₁₀ CFU/g) |
| 0 | 7.04 ± 0.13* | 6.29 ± 0.01* |
| 20 | 0.00 | 0.00 |
| (700-800 | | |
| mJ/sqcm) | | |
| 40 | 0.00 | 0.00 |
| (1400-1600 | | |
| mJ/sqcm) | | |

*Average ± standard deviation for three replications is given. Based on UV fluence measurements with SED240 detector, 20 s correspond to 700-800 mJ/cm² and 40 s correspond to 1400-1600 mJ/cm². All these values represent complete inactivation. Concentrations of original inoculum were 8.90 ± 0.12 log₁₀ CFU/mL for E. coli O157:H7 and 8.32 ± 0.09 log₁₀ CFU/mL for Geobacillus stearothermophillus spores, respectively.

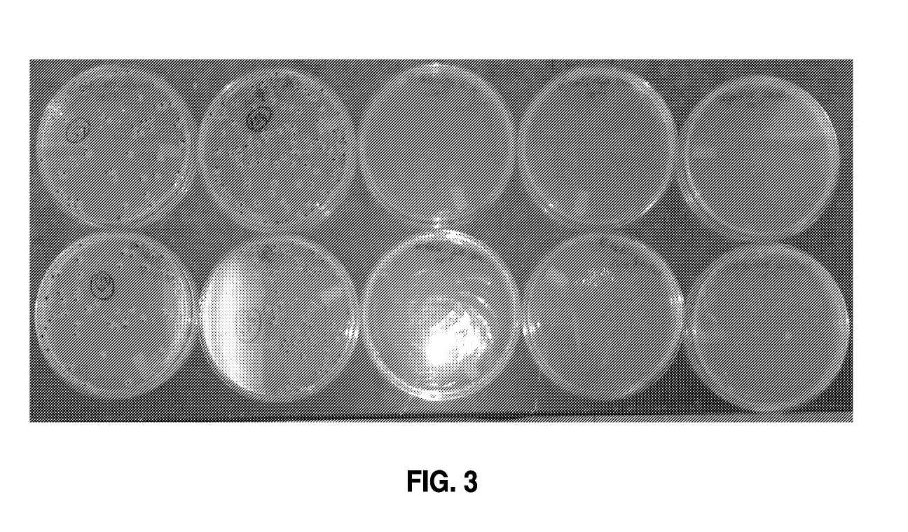

FIG. 3 is a photograph of test Soy Agar plates, right three columns showing no bacterial growth with pulsed light treatment of the invention, and left two columns being control plates showing positive EColi growth.

### Rheology tests

Frequency sweep rheology experiments were performed to provide an indication of gel stability after the present sterilization methods. The elastic modulus in shear (G′) under dynamic frequency can be compared for various treatment conditions.

When heat sterilized using conventional autoclave procedures (120 C, 30 min), samples show about a 40% drop in G′, indicating gel structure destruction.

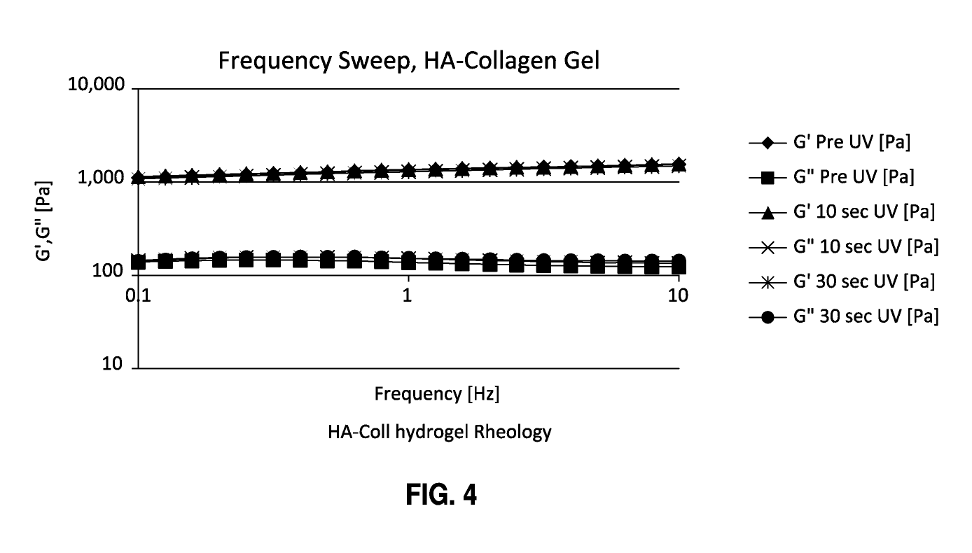

FIG. 4 shows a rheology plot for the HA/Coll gel, control (no pulsed light treatment), 10 second pulsed light treatment in accordance with the invention, and 30 second pulsed light treatment in accordance with the invention:
i. G′and G″ for pre-pulsed light treatment (“Pre UV”)
ii. G′and G″ for 10 sec light treatment (“10 sec UV”)
iii. G′and G″ for 30 sec light treatment (“30 sec UV”)
iv. The untreated control, 30 sec pulsed light-treated and 10 sec pulsed light-treated samples show change in modulus in the range of between about 5% to about 8%. This marginal change indicates minimal damage to the structure of hydrogel.

### EXAMPLE 2

### Hyaluronic acid-based Hydrogel Material

The hydrogel material tested in this experiment was commercial HA-based dermal filler product (hereinafter sometimes “HA gel”) marketed under the trademark Juvederm®, manufactured by Allergan, Inc. (IRVINE, Calif.).

1 g of inoculums was added to 5 g of HA gel to yield approximately 6 to 7 logᵢₙ CFU/g. The inoculated hydrogel samples were packaged in HDPE bags, made into thin pouches and sealed with the whirl-pak for the pulsed light treatment.

The same equipment described in Example 1 was used for providing the radiation treatment.

Specific conditions for the HA gel treatment were as follows:

Treatment times: 1, 2, 5, and 10 seconds. Distance from pulsed light source: 3.26″ from the quartz window. Sample weight: 5 g.

Microorganisms: E. coli 0157:H7 cells and G. stearothermophilus spores. Replications:

Treatment: Only one side of the package was treated with pulsed light for the required amount of time.

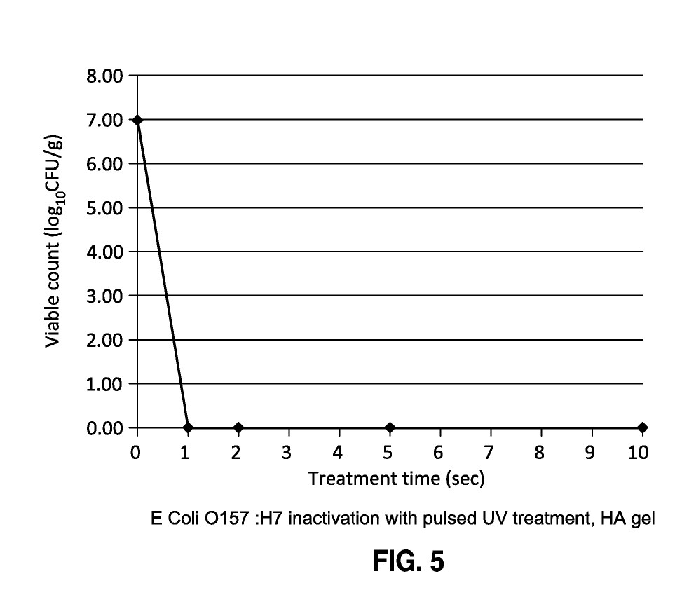

Pulsed light treatment was effective in inactivation of the bacteria, as shown in Table 2 below and in FIG. 5.

TABLE 2
E coli O157:H7 survival in HA gel
treated with pulsed UV treatment

| Treatment | Replication 1 | Replication 2 | Replication 3 | Average |
|---|---|---|---|---|
| time | (log₁₀ | (log₁₀ | (log₁₀ | (log₁₀ |
| (sec) | CFU/g) | CFU/g) | CFU/g) | CFU/g)³ |
| 0⁴ | 6.94 | 6.98 | 7.01 | 6.98 ± 0.03 |
| 1 | 0.00 | 0.00 | 0.00 | 0.00 ± 0.00 |
| 2 | 0.00 | 0.00 | 0.00 | 0.00 ± 0.00 |
| 5 | 0.00 | 0.00 | 0.00 | 0.00 ± 0.00 |
| 10 | 0.00 | 0.00 | 0.00 | 0.00 ± 0.00 |

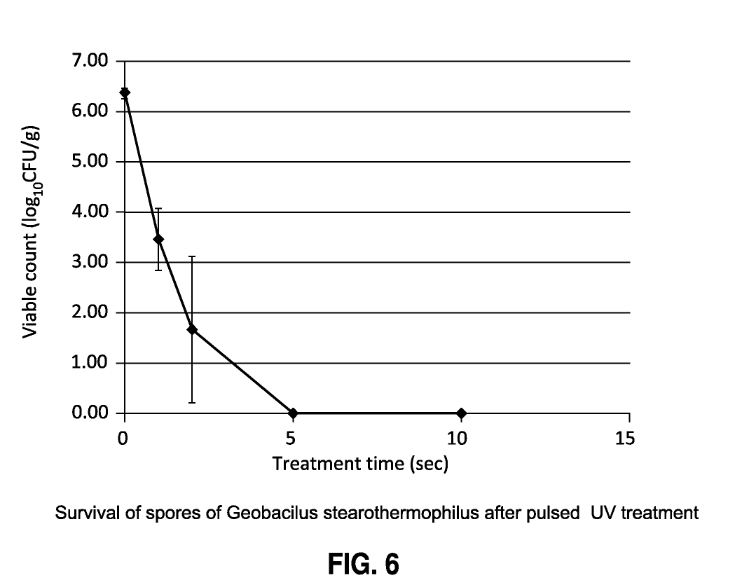

Spores are more resistant than cells. Lower treatment times showed some variations with Geobacilus stearothemophilus. This is shown in Table 3 and FIG. 6.

TABLE 3
Spores of Geobacilus stearothermophilus
after pulsed UV treatment of HA gel

| Treatment | Replication 1 | Replication 2 | Replication 3 | Average |
|---|---|---|---|---|
| time | (log₁₀ | (log₁₀ | (log₁₀ | (log₁₀ |
| (sec) | CFU/g) | CFU/g) | CFU/g) | CFU/g)³ |
| 0⁴ | 6.49 | 6.37 | 6.27 | 6.38 ± 0.11 |
| 1 | 3.04 | 4.16 | 3.18 | 3.46 ± 0.61 |
| 2 | 2.30 | 2.70 | 0.00 | 1.67 ± 1.46 |
| 5 | 0.00 | 0.00 | 0.00 | 0.00 ± 0.00 |
| 10 | 0.00 | 0.00 | 0.00 | 0.00 ± 0.00 |

The results indicated that pulsed light treatment of the HA gel was effective and produced log 6 reduction in bacterial cells and spores in very short amount of time, in this case, 10 seconds.

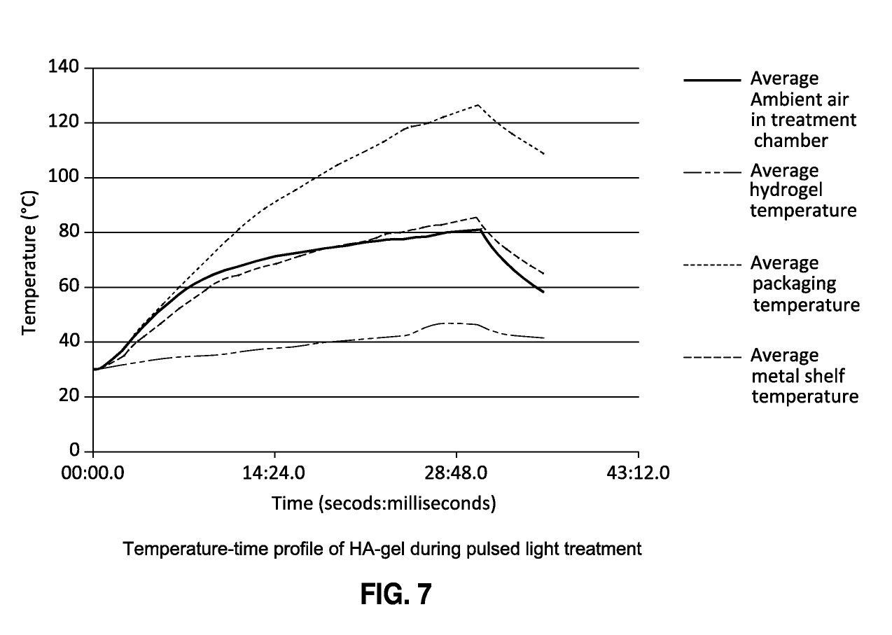

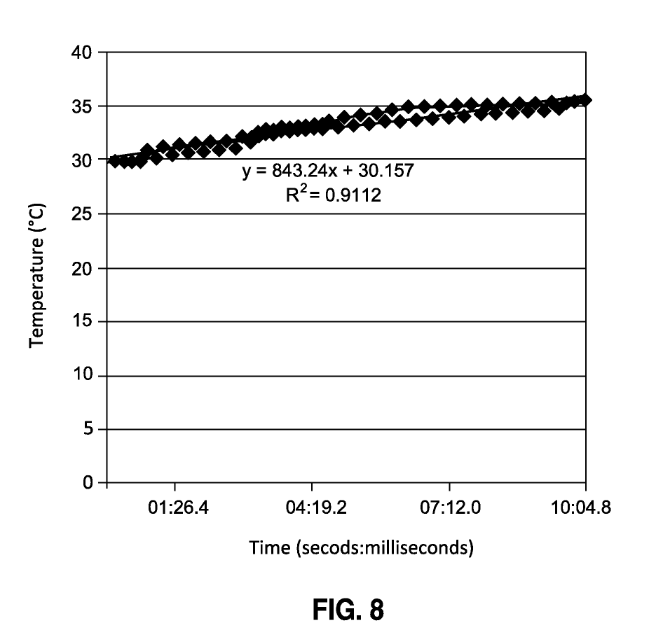

Further, the temperature of the hydrogel did not increase significantly during the pulsed light treatment. For instance, a 10 second treatment resulted in approximately 5° C. temperature increase as shown in FIG. 7. A maximum temperature increase of 19° C. was observed after 30 second treatment. The temperature increase is linear during the tested treatment conditions. FIG. 8 shows a plot of temperature increase over time for the hydrogels tested. Here, a 10 minute interval shows a linear correlation between temperature and pulsed light treatment time.

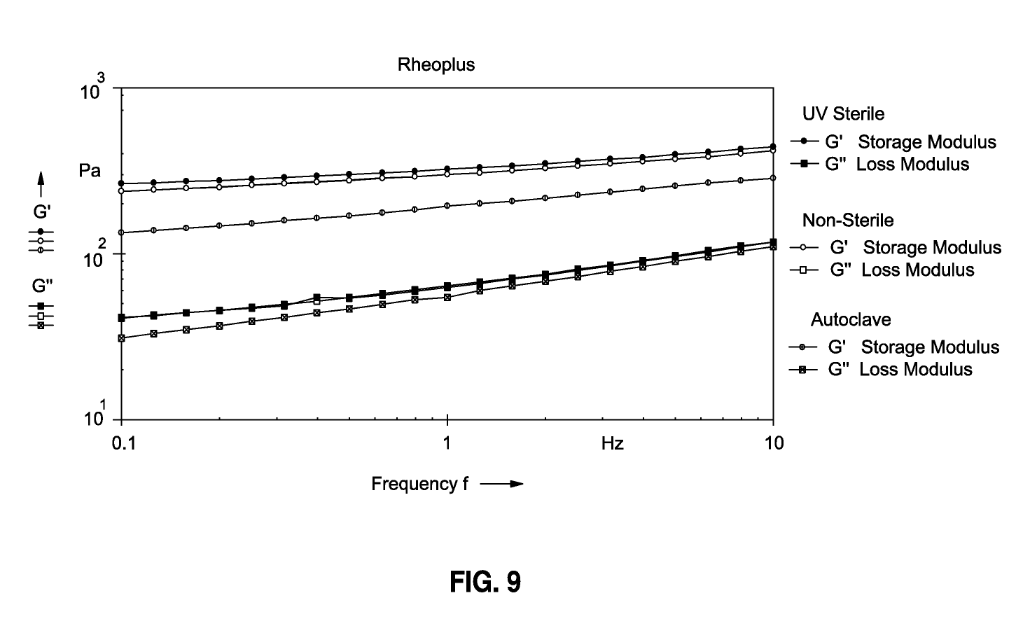

FIG. 9 shows comparative frequency sweep rheology characterization of the HA gel sterilized with pulsed light in accordance with the invention, the HA gel without sterilization treatment, and the HA gel treated with conventional heat sterilization techniques, in this case, conventional autoclave sterilization. As shown, the HA gel loses almost 30% of G′, or rheology shear modulus, when heat sterilized. In comparison, the HA gel maintains both G′ and G″ when sterilized using pulsed light in accordance with the invention. It can be concluded from this data that pulsed-light treated and untreated HA gel, which had a change in rheological properties within 5% to 8%, is insignificant, relative to conventional heat-sterilized HA gel.

While this invention has been described with respect to various specific examples and embodiments, it is to be understood that the invention is not limited thereto and that it can be variously practiced within the scope of the invention.

What is claimed is:

1. A method for sterilizing a gel composition, the method comprising:
subjecting the composition to pulsed light comprising broadband spectrum radiation having a band range from about 100 nm to about 1100 nm wavelength, the pulsed light being effective to sterilize the composition without causing significant change in rheology of the composition.

2. The method of claim 1 wherein the pulsed light has an energy defined by a UV fluence at 254 nm of between about 100 mJ/sqcm to about 2000 mJ/sqcm.

3. The method of claim 1 wherein the pulsed light has an energy defined by a UV fluence at 254 nm of between about 300 mJ/sqcm to about 1800 mJ/sqcm.

4. The method of claim 1 wherein the pulsed light has an energy defined by a UV fluence at 254 nm of between about 700 mJ/sqcm to about 800 mJ/sqcm.

5. The method of claim 1 wherein the pulsed light has an energy defined by a UV fluence at 254 nm of between about 1400 mJ/sqcm to about 1600 mJ/sqcm.

6. The method of claim 1 wherein radiation is in the form of pulsed radiation having a pulse frequency of between about 1 pulse per second to about 10 pulses per second.

7. The method of claim 1 wherein the composition is subjected to the dose for a time period of no greater than 240 seconds.

8. The method of claim 1 wherein the composition is subjected to the pulsed light for a time period of no greater than 120 seconds.

9. The method of claim 1 wherein the composition is subjected to the pulsed light for a time period of no greater than 40 seconds.

10. The method of claim 1 wherein the composition is subjected to the pulsed light for a time period of no greater than 30 seconds.

11. The method of claim 1 wherein the composition is subjected to the pulsed light for a time period of no greater than one second to 20 seconds.

12. The method of claim 1 wherein the composition is subjected to the pulsed light for a time period of no greater than 10 seconds.

13. The method of claim 1 wherein the composition is subjected to the pulsed light for a time period of no greater than 5 seconds.

14. The method of claim 1 wherein the composition is subjected to the pulsed light for a time period of no greater than one second.

15. The method of claim 1 wherein the broadband spectrum radiation has a wavelength distribution of about 54% UV wavelengths, 26% visible wavelengths and about 20% infrared wavelengths.

16. The method of claim 1 wherein the pulsed light is provided by a Xenon lamp.

17. The method of claim 1 wherein the composition comprises collagen.

18. The method of claim 1 wherein the composition comprises hyaluronic acid.

19. The method of claim 1 wherein the composition comprises hyaluronic acid and collagen.

20. The method of claim 1 wherein the composition is a gel comprising hyaluronic acid and collagen.

21. The method of claim 1 wherein the pulsed light is effective to sterilize the composition without raising the temperature of the composition more than 90 degrees C.

22. The method of claim 1 wherein the pulsed light is effective to sterilize the composition without raising the temperature of the composition more than 20 degrees C.

23. The method of claim 1 wherein the pulsed light is effective to sterilize the composition without raising the temperature of the composition more than 15 degrees C.

24. The method of claim 1 wherein the pulsed light is effective to sterilize the composition without raising the temperature of the composition more than 10 degrees C.

25. The method of claim 1 wherein the pulsed light is effective to sterilize the composition without raising the temperature of the composition more than 5 degrees C.

26. A product comprising crosslinked hyaluronic acid and collagen, sterilized by the method of claim 1.

27. The method of claim 1 wherein the pulsed light is effective to sterilize the composition with a loss in Rheology (G′/G″) of less than about 8%.

28. The method of claim 1 wherein the pulsed light is effective to sterilize the composition with a loss in Rheology (G′/G″) of less than about 5%.

29. A product useful for combining with adipose tissue for use in fat grafting procedures, the product comprising:
a composition comprising crosslinked hyaluronic acid and collagen;
the composition having been sterilized by subjecting the composition to pulsed light comprising broadband spectrum radiation having a band range from about 100 nm to about 1100 nm wavelength, the pulsed light being effective to sterilize the composition without causing significant change in rheology of the composition.

30. The product of claim 29 further comprising a vial or syringe containing the composition.

31. The product of claim 29 wherein the composition has been so subjected to the pulsed light while the composition was contained in the vial or the syringe.
